(19) United States
(12) Patent Application Publication
(10) Pub. No.: US 2026/0365613 A1
(43) Pub. Date: Nov. 19, 2026

(54) PRODUCTION OF CYANO-CONTAINING COMPOUNDS

(71) Applicant: INV NYLON CHEMICALS AMERICAS, LLC, Wilmington, DE (US)

(72) Inventors: Benjamin David Herzog, Wichita, KS (US); Milind V. Kantak, Wilmington, DE (US)

(21) Appl. No.: 19/121,145

(22) PCT Filed: Oct. 19, 2023
(86) PCT No.: PCT/IB2023/060558

§ 371 Date: Apr. 15, 2025

Related U.S. Application Data
(60) Provisional application No. 63/419,352, filed on Oct. 26, 2022.

(51) Int. Cl. C07C 253/26; C01C 3/02; C07C 253/34

(52) U.S. Cl. CPC C07C 253/26; C01C 3/02; C07C 253/34

(57) ABSTRACT

In a method of producing cyano-containing compounds, ammonia, a source of oxygen and an organic compound are reacted in an ammoxidation reactor to produce a reaction product comprising a target cyano-containing compound selected from hydrogen cyanide and an organonitrile compound. The reaction product is supplied to a separation section to recover at least part of the target compound. A waste stream comprising at least one further organonitrile compound different from the target compound is combined with at least one organic oxygenate diluent compatible with an ammoxidation reaction to produce a diluted waste stream and the diluted waste stream is supplied to the ammoxidation reactor.

### CROSS-REFERENCE TO RELATED APPLICATION

This application claims the benefit of priority to U.S. Provisional Patent Application Ser. No. 63/419,352 entitled “PRODUCTION OF CYANO-CONTAINING COMPOUNDS,” filed Oct. 26, 2022, the disclosure of which is incorporated herein in its entirety by reference.

### FIELD

The present disclosure relates to the production of cyano-containing compounds, specifically hydrogen cyanide and organonitriles, and in particular to a method of producing such compounds with improved yield and/or reduced nitrogen oxide [NOₓ] emissions.

### BACKGROUND

One widely practiced method of producing cyano-containing compounds is “ammoxidation”, wherein C₁ hydrocarbons, C₃ hydrocarbons, alcohols, carboxylic acids, ketones, low-molecular weight polyols, tetrahydrofuran and other organic compounds undergo a chemical transformation in the presence of ammonia and an oxygen source to form hydrogen cyanide [HCN] and/or other organonitrile compounds. Well-known examples include the SOHIO ammoxidation process that converts propylene or propane to acrylonitrile and the Andrussow process wherein HCN is formed from methane (natural gas), ammonia and an oxygen source.

During the production of any given target cyano-containing compound by ammoxidation, several byproduct organonitriles, referred to as “ON byproducts” are inevitably produced. For example, the SOHIO ammoxidation process produces acetonitrile and propionitrile in addition to acrylonitrile and HCN. It is industrially known and observed that such ON byproducts can result in equipment fouling, plugging, corrosion and foaming as a result of degradation/polymerization, particularly in the product separation and recovery sections. All of this complicates the product recovery/separation process. Further, unless removed, these ON byproducts can carry into the finished products as unwanted impurities.

The problem of ON byproduct accumulation in nitrile fractionation equipment has conventionally been addressed by allowing the ONs to build up at an identified tray (stage) or section in a fractionation tower. The ONs are then periodically purged from that tray (stage) or section and burned as fuel or processed in thermal oxidizers. The problem is that this downgrades the value of this ON stream to fuel value. Thermal oxidation of such nitrogen-containing purge streams also leads to increased nitrogen oxides [NOₓ] emissions to the environment. While it would be desirable to recycle these purged ONs to the process feed, nitrile production facilities are not designed for wide variations in process feed composition.

Therefore, there continues to be an industrial need for developing an improved large-scale nitriles production process that handles the organonitrile byproduct streams in a way to upgrade their value while reducing or even eliminating nitrogen oxide [NOₓ] emissions resulting therefrom.

### SUMMARY

According to the present disclosure, it has now been found that, by combining the ON byproducts removed from the separation system of an ammoxidation process with an oxygenate diluent, the byproducts can be recycled to the process with maintaining stable operating control, thereby increasing the value of the byproduct stream while reducing NOₓ emissions.

Thus, in one aspect, the present application provides a method of producing cyano-containing compounds, the method comprising the steps of:
(a) reacting ammonia, a source of oxygen and an organic compound in an ammoxidation reactor to produce a reaction product comprising a target cyano-containing compound selected from hydrogen cyanide and an organonitrile compound;
(b) supplying the reaction product to a separation section to recover at least part of the target compound;
(c) providing a waste stream comprising at least one further organonitrile compound different from the target compound;
(d) combining at least part of the waste stream with at least one organic oxygenate diluent compatible with an ammoxidation reaction to produce a diluted waste stream; and
(e) supplying the diluted waste stream to the ammoxidation reactor.

### BRIEF DESCRIPTION OF THE DRAWINGS

FIG. 1 is a schematic representation of a conventional co-production facility 100 for making acrylonitrile [ACRN] and HCN.

FIG. 2 is a schematic representation of a co-production facility 200 for making acrylonitrile [ACRN] and HCN according to a first embodiment of the present disclosure.

FIG. 3 is a schematic representation of a co-production facility 300 for making acrylonitrile [ACRN] and HCN according to second embodiment of the present disclosure.

FIG. 4 is a schematic representation of a production facility 400 for making acrylonitrile [ACRN] according to a third embodiment of the present disclosure.

It is to be noted that, in all Figures herein, there are many details around the various flow lines [process, utility, bypass, vents, purges, sampling], instrumentations, controls, pumps, valves, etc. that are known to a person skilled in the art, and therefore, not shown.

### DETAILED DESCRIPTION

As used herein, the terms “Acrylo” or “ACRN” refer to acrylonitrile and can be used interchangeably.

As used herein, the terms “Aceto” or “ACN” refer to acetonitrile and can be used interchangeably.

As used herein, the term “HCN” refers to hydrogen cyanide.

As used herein, the term “target compound” or “target compounds” refers to the specific cyano-compound or compounds selected from hydrogen cyanide and organonitrile compounds desired to be produced by the method described herein. Examples target compounds of industrial utility may include, but are not limited to, hydrogen cyanide and C₁-C₆ nitriles and dinitriles, such as acetonitrile, acrylonitrile, succinonitrile, adiponitrile, methylglutaronitrile, pentenenitrile, and glutaronitrile.

As referred to herein, the terms “nitriles production process” or “nitriles production facility” refer to a chemical production process or facility where useful nitriles are manufactured. Non-limiting examples of nitrile production process are Andrussow HCN process, BMA (Degussa) HCN process, Shawinigan HCN process, methanol ammoxidation to HCN process; ethanol ammoxidation to acetonitrile process, succinonitrile from heterocyclic materials [tetrahydrofuran for example], glycerol ammoxidation to nitriles, ammoxidation of propylene and/or propane acrylonitrile process, and ammoxidation acrylonitrile process with methanol co-feed to produce additional HCN.

As used herein, the term “ammoxidation” refers to a well-known chemical synthesis step wherein C₁ hydrocarbons, C₃ hydrocarbons, alcohols, carboxylic acids, ketones, low-molecular weight polyols [glycerol], tetrahydrofuran [THF], acetonitrile and such undergoes a chemical transformation in the presence of ammonia and an oxygen source to form nitriles (represented by a functional group “—C≡N” or “—CN”).

As used herein, the term “Andrussow” refers to a well-known chemical synthesis step wherein HCN is formed from methane (natural gas), ammonia and an oxygen source. Depending on the oxygen content, Andrussow process can be labeled as air-Andrussow, an air-rich or enriched-air Andrussow or 100% oxygen-feed Andrussow process.

As used herein, the interchangeable terms “organonitrile-containing waste stream” or “organonitrile byproduct stream” or “byproduct ONs” refer to a stream or streams comprising nitrile-containing organic components different from the target compound(s) that are considered as undesirable impurities to be removed from the nitriles process. In some embodiments, the organonitrile impurities have a higher molecular weight, such as at least 10% higher, than the target cyano compound. It will, of course, be appreciated that an organonitrile that may be the desired target compound in one process may be considered an undesirable byproduct in another process.

In one embodiment, the organonitrile-containing waste streams may be available from an adiponitrile production facility. Additional ON components present in such waste streams may include, and not limited to, unsaturated or saturated C₃-C₄ nitriles and dinitriles, linear or branched pentenenitriles, 2-methylglutaronitrile (MGN), methylene-glutaronitrile, ethyl succinonitrile (ESN) and adiponitrile (ADN), merely to name a few examples. Such organonitrile-containing waste streams may be concentrated from the adiponitrile recovery/purification step and used according to the present disclosure.

In acrylonitrile production, undesirable byproduct ONs include hydrogen cyanide, acetonitrile, propionitrile, methacrylonitrile, nitrile dimers, trimers and oligomers, butanenitriles, 6-aminocapronitrile, 4-amino-2-methyl-5,6-trimethylene pyrimidine, cyclic nitriles, and polymers of acrylonitrile.

In HCN production, undesired byproduct ONs include acetonitrile, propionitrile, acrylonitrile, half-half nitrile-acid, nitrile dimers, trimers and oligomers, and butanenitriles.

Table 1 provides a list of likely undesired byproduct ONs depending on the desired target product(s) made from conventional manufacturing processes.

TABLE 1
Undesired ONs Generated in various Nitriles Production Processes

| | | Undesired Organo-nitriles [ONs] generated in the process | | | | | | | |
|---|---|---|---|---|---|---|---|---|---|
| | | Hydrogen | Aceto- | Propio- | Acrylo- | Methacrylo- | Nitrile | Nitrile | Nitrile |
| Desired Nitrile Product | Process(es) | Cyanide | nitrile | nitrile | nitrile | nitrile | Dimers | Trimers | Oligomers |
| Hydrogen Cyanide | Andrussow [O2/Air] | | x | x | x | | x | x | x |
| | BMA (Degussa) | | x | x | x | | x | x | x |
| | Shawinigan | | x | x | x | | x | x | x |
| | Ammoxidation of various | | x | x | x | | x | x | x |
| | feeds [alcohol, ketone, acid, | | | | | | | | |
| | nitrile] | | | | | | | | |
| | By-product from | | x | x | x | x | x | x | x |
| | Propane/Propylene | | | | | | | | |
| | Ammoxidation | | | | | | | | |
| Acetonitrile | Alcohol Ammoxidation | x | | x | x | | x | x | x |
| | By-product from | x | | x | x | x | x | x | x |
| | Propane/Propylene | | | | | | | | |
| | Ammoxidation | | | | | | | | |
| Acrylonitrile | Propane/Propylene | x | x | x | | x | x | x | x |
| | Ammoxidation | | | | | | | | |
| Succinonitrile | from acrylonitrile & HCN | x | x | x | x | x | x | x | x |
| | from ketone cyanohydrins | x | x | x | | | x | x | x |
| | from C4 Heterocyclics [THF] | | | | | | x | x | x |
| Linear/Branched | Olefin Hydrocyanation; | x | | x | | | x | x | x |
| Pentenenitriles | Isomerization | | | | | | | | |
| Adiponitrile | Olefin Hydrocyanation | x | | x | | | x | x | x |
| | Acrylonitrile Dimerization - | x | | x | x | x | x | x | x |
| | electrochemical coupling | | | | | | | | |
| | Acrylonitrile Dimerization - | x | | x | x | | x | x | x |
| | catalytic chemial coupling | | | | | | | | |
| | Adipic Acid Ammoxidation | x | | x | | | x | x | x |
| | Ammoxidation of Adipic Acid | x | | x | | | x | x | x |
| | made from non-fossil | | | | | | | | |
| | materials | | | | | | | | |
| | cyclohexane/cyclohexene/ | x | | x | | | x | x | x |
| | cyclohexanol/Hexanediol | | | | | | | | |
| | Ammoxidation | | | | | | | | |
| Methyl Glutaronitrile | Olefin Hydrocyanation | x | | x | | | x | x | x |
| Ethyl Succinonitrile | Olefin Hydrocyanation | x | | x | | | x | x | x |
| Polymer from C₅ diamine | C5 diamine obtained from | | | | | | | | |
| | fermentation or chemical | | | | | | | | |
| | route using non-fossil | | | | | | | | |
| | material | | | | | | | | |

| | | Undesired Organo-nitriles [ONs] generated in the process | | | | | | | |
|---|---|---|---|---|---|---|---|---|---|
| | | | | | 2,3,4,5- | | | | |
| | | | Acrylo- | | tetra- | | | | |
| | | half-half | nitrile | | hydropy- | | Amino- | | |
| | | Nitrile- | Hydro- | | ridine | Piper- | capro- | Succino- | Butane- |
| Desired Nitrile Product | Process(es) | acid | trimer | Pyridine | [THP] | idine | lactam | nitrile | nitriles |
| Hydrogen Cyanide | Andrussow [O2/Air] | | | | | | | | |
| | BMA (Degussa) | | | | | | | | |
| | Shawinigan | | | | | | | | |
| | Ammoxidation of various | x | | | | | | | |
| | feeds [alcohol, ketone, acid, | | | | | | | | |
| | nitrile] | | | | | | | | |
| | By-product from | | | | | | | | x |
| | Propane/Propylene | | | | | | | | |
| | Ammoxidation | | | | | | | | |
| Acetonitrile | Alcohol Ammoxidation | x | | | | | | | |
| | By-product from | | | | | | | | x |
| | Propane/Propylene | | | | | | | | |
| | Ammoxidation | | | | | | | | |
| Acrylonitrile | Propane/Propylene | | | | | | | | x |
| | Ammoxidation | | | | | | | | |
| Succinonitrile | from acrylonitrile & HCN | x | | | | | | | x |
| | from ketone cyanohydrins | x | | | | | | | x |
| | from C4 Heterocyclics [THF] | x | x | x | | | | | x |
| Linear/Branched | Olefin Hydrocyanation; | | | | | | | | x |
| Pentenenitriles | Isomerization | | | | | | | | |
| Adiponitrile | Olefin Hydrocyanation | | | | | | | | |
| | Acrylonitrile Dimerization - | | x | | | | | x | |
| | electrochemical coupling | | | | | | | | |
| | Acrylonitrile Dimerization - | | x | | | | | x | |
| | catalytic chemial coupling | | | | | | | | |
| | Adipic Acid Ammoxidation | x | | | | | | x | |
| | Ammoxidation of Adipic Acid | x | | | | | | x | |
| | made from non-fossil | | | | | | | | |
| | materials | | | | | | | | |
| | cyclohexane/cyclohexene/ | x | | | | | | x | |
| | cyclohexanol/Hexanediol | | | | | | | | |
| | Ammoxidation | | | | | | | | |
| Methyl Glutaronitrile | Olefin Hydrocyanation | x | | | | | | | x |
| Ethyl Succinonitrile | Olefin Hydrocyanation | x | | | | | | x | x |
| Polymer from C₅ diamine | C5 diamine obtained from | | | x | x | x | x | | |
| | fermentation or chemical | | | | | | | | |
| | route using non-fossil | | | | | | | | |
| | material | | | | | | | | |

| | | Undesired Organo-nitriles [ONs] generated in the process | | | | | | | |
|---|---|---|---|---|---|---|---|---|---|
| | | | Valero- | Linear/ | | | | | |
| | | | nitrile | Branched | Methyl | Ethyl- | Methylene | | |
| | | Butene- | [Pentane- | Pentene- | Glutaro- | succino- | Glutaro- | Adipo- | adip- |
| Desired Nitrile Product | Process(es) | nitriles | nitrile] | nitriles | nitrile | nitrile | nitrile | nitrile | amide |
| Hydrogen Cyanide | Andrussow [O2/Air] | | | | | | | | |
| | BMA (Degussa) | | | | | | | | |
| | Shawinigan | | | | | | | | |
| | Ammoxidation of various | | | | | | | | |
| | feeds [alcohol, ketone, acid, | | | | | | | | |
| | nitrile] | | | | | | | | |
| | By-product from | | | | | | | | |
| | Propane/Propylene | | | | | | | | |
| | Ammoxidation | | | | | | | | |
| Acetonitrile | Alcohol Ammoxidation | | | | | | | | |
| | By-product from | | | | | | | | |
| | Propane/Propylene | | | | | | | | |
| | Ammoxidation | | | | | | | | |
| Acrylonitrile | Propane/Propylene | | | | | | | | |
| | Ammoxidation | | | | | | | | |
| Succinonitrile | from acrylonitrile & HCN | | | | | | | | |
| | from ketone cyanohydrins | | | | | | | | |
| | from C4 Heterocyclics [THF] | | | | | | | | |
| Linear/Branched | Olefin Hydrocyanation; | | x | | | | | | |
| Pentenenitriles | Isomerization | | | | | | | | |
| Adiponitrile | Olefin Hydrocyanation | x | | x | x | x | | | |
| | Acrylonitrile Dimerization - | | | | x | x | x | | |
| | electrochemical coupling | | | | | | | | |
| | Acrylonitrile Dimerization - | | | | x | | x | | |
| | catalytic chemial coupling | | | | | | | | |
| | Adipic Acid Ammoxidation | | | | x | x | | | x |
| | Ammoxidation of Adipic Acid | | | | x | x | | | x |
| | made from non-fossil | | | | | | | | |
| | materials | | | | | | | | |
| | cyclohexane/cyclohexene/ | | | | | | | | x |
| | cyclohexanol/Hexanediol | | | | | | | | |
| | Ammoxidation | | | | | | | | |
| Methyl Glutaronitrile | Olefin Hydrocyanation | x | | x | | x | | x | |
| Ethyl Succinonitrile | Olefin Hydrocyanation | x | | x | x | | | x | |
| Polymer from C₅ diamine | C5 diamine obtained from | | | | | | | | |
| | fermentation or chemical | | | | | | | | |
| | route using non-fossil | | | | | | | | |
| | material | | | | | | | | |

| | | Undesired Organo-nitriles [ONs] generated in the process | | | | | |
|---|---|---|---|---|---|---|---|
| | | | | Hydroxy | 2-Cyanocyclo- | | |
| | | | Unsaturated | propio- | pentyl- | Cyano- | Cyano- |
| | | adipamic | C6 | nitrile | idenimine | valeric | valer- |
| Desired Nitrile Product | Process(es) | acid | Dinitriles | [HOPN] | [CPI] | acid | amide |
| Hydrogen Cyanide | Andrussow [O2/Air] | | | | | | |
| | BMA (Degussa) | | | | | | |
| | Shawinigan | | | | | | |
| | Ammoxidation of various | | | | | | |
| | feeds [alcohol, ketone, acid, | | | | | | |
| | nitrile] | | | | | | |
| | By-product from | | | | | | |
| | Propane/Propylene | | | | | | |
| | Ammoxidation | | | | | | |
| Acetonitrile | Alcohol Ammoxidation | | | | | | |
| | By-product from | | | | | | |
| | Propane/Propylene | | | | | | |
| | Ammoxidation | | | | | | |
| Acrylonitrile | Propane/Propylene | | | | | | |
| | Ammoxidation | | | | | | |
| Succinonitrile | from acrylonitrile & HCN | | | | | | |
| | from ketone cyanohydrins | | | | | | |
| | from C4 Heterocyclics [THF] | | | | | | |
| Linear/Branched | Olefin Hydrocyanation; | | | | | | |
| Pentenenitriles | Isomerization | | | | | | |
| Adiponitrile | Olefin Hydrocyanation | | | | x | | |
| | Acrylonitrile Dimerization - | | x | x | x | | |
| | electrochemical coupling | | | | | | |
| | Acrylonitrile Dimerization - | | x | | x | | |
| | catalytic chemial coupling | | | | | | |
| | Adipic Acid Ammoxidation | x | | | x | x | x |
| | Ammoxidation of Adipic Acid | x | | | x | x | x |
| | made from non-fossil | | | | | | |
| | materials | | | | | | |
| | cyclohexane/cyclohexene/ | x | | | x | x | x |
| | cyclohexanol/Hexanediol | | | | | | |
| | Ammoxidation | | | | | | |
| Methyl Glutaronitrile | Olefin Hydrocyanation | | | | | | |
| Ethyl Succinonitrile | Olefin Hydrocyanation | | | | | | |
| Polymer from C₅ diamine | C5 diamine obtained from | | | | | | |
| | fermentation or chemical | | | | | | |
| | route using non-fossil | | | | | | |
| | material | | | | | | |

| | | Undesired Organo-nitriles [ONs] generated in the process | | | | | |
|---|---|---|---|---|---|---|---|
| | | | | | 4-amino- | | |
| | | | | | 2-methyl- | | |
| | | Tricyano- | Cyano- | 6-amino- | 5,6-trimethy- | | Polymer |
| | | hexane | ethylated | capro- | lene | Cyclic | of Acrylo- |
| Desired Nitrile Product | Process(es) | [TCH] | species* | nitrile | pyrimidine | Nitriles | nitrile |
| Hydrogen Cyanide | Andrussow [O2/Air] | | | | | | |
| | BMA (Degussa) | | | | | | |
| | Shawinigan | | | | | | |
| | Ammoxidation of various | | | x | x | x | |
| | feeds [alcohol, ketone, acid, | | | | | | |
| | nitrile] | | | | | | |
| | By-product from | | | x | x | x | x |
| | Propane/Propylene | | | | | | |
| | Ammoxidation | | | | | | |
| Acetonitrile | Alcohol Ammoxidation | | | | | | |
| | By-product from | | | x | x | x | x |
| | Propane/Propylene | | | | | | |
| | Ammoxidation | | | | | | |
| Acrylonitrile | Propane/Propylene | | | x | x | x | x |
| | Ammoxidation | | | | | | |
| Succinonitrile | from acrylonitrile & HCN | | | x | x | x | x |
| | from ketone cyanohydrins | | | x | x | x | |
| | from C4 Heterocyclics [THF] | | | | | | |
| Linear/Branched | Olefin Hydrocyanation; | | | | | x | |
| Pentenenitriles | Isomerization | | | | | | |
| Adiponitrile | Olefin Hydrocyanation | | | | | x | |
| | Acrylonitrile Dimerization - | x | x | | | x | x |
| | electrochemical coupling | | | | | | |
| | Acrylonitrile Dimerization - | x | | | | x | x |
| | catalytic chemial coupling | | | | | | |
| | Adipic Acid Ammoxidation | | | x | x | x | |
| | Ammoxidation of Adipic Acid | | | x | x | x | |
| | made from non-fossil | | | | | | |
| | materials | | | | | | |
| | cyclohexane/cyclohexene/ | | | x | x | x | |
| | cyclohexanol/Hexanediol | | | | | | |
| | Ammoxidation | | | | | | |
| Methyl Glutaronitrile | Olefin Hydrocyanation | | | | | x | |
| Ethyl Succinonitrile | Olefin Hydrocyanation | | | | | x | |
| Polymer from C₅ diamine | C5 diamine obtained from | | | | x | x | |
| | fermentation or chemical | | | | | | |
| | route using non-fossil | | | | | | |
| | material | | | | | | |

*Cyano-ethylated species may include Bis-cyanoethylether [BCE], Mono-cyanoethylpropyl amine [MCPA]

As shown in the last row of Table 1, undesired byproduct ONs, generated during the process of making polymers from bio-sourced C5 diamine [pentamethylene diamine], may be useful in the disclosed process.

In conventional ammoxidation processes, these byproduct ONs are carried through the product recovery/separation/purification steps and accumulate/concentrate in the downstream refining equipment (recovery-separation columns, HCN product refining train, decanters, etc.). In addition, byproduct ONs are often unsaturated or will otherwise self-polymerize (forming monomers, dimers, trimers, etc.) and are susceptible to building molecular weight under separation conditions.

A conventional way to manage/control these byproduct ONs is to withdraw purge streams from appropriate locations where these ONs are most concentrated and to remove them from the process. Often this purge is discontinuous to minimize the loss of useful products. This practice causes intermediate boiling ONs to accumulate impacting unit performance.

The above-mentioned organonitrile purge streams obtained from nitriles processes are either removed via off-gas and/or disposed-off as concentrated liquid stream. The most common method for disposing such organic purge streams is by thermal destruction in thermal oxidizers [TOs]. Thermal destruction of such nitrogen-containing purge streams is disadvantageous not only because it downgrades the value of the streams but also potentially leads to increased nitrogen oxides [NOₓ] emissions to the environment.

To address this problem, the present disclosure provides a method of producing a cyano-containing compound selected from hydrogen cyanide and an organonitrile compound, in which ammonia, a source of oxygen and an organic compound are reacted in an ammoxidation reactor to produce a reaction product comprising the target cyano-containing compound. The reaction product is then supplied to a separation section where at least part of the target compound is recovered. A waste stream comprising at least one further organonitrile compound different from the target compound is combined with at least one organic oxygenate diluent compatible with an ammoxidation reaction to produce a diluted waste stream and the diluted waste stream is supplied to the ammoxidation reactor. Generally, the waste stream will comprise an organonitrile byproduct-containing stream removed from the separation system used to recover the target cyano-containing compound. However, in some embodiments, at least part of the waste stream may be supplied from a source different from the separation system used to recover the target cyano-containing compound.

The organic compound fed to ammoxidation reactor will depend on the target cyano-containing compound to be produced. For example, when the target compound is HCN, the organic compound fed to ammoxidation reactor may be methane, whereas when the target compound is acrylonitrile of acetonitrile, the organic compound may be propylene and/or propane. Other suitable organic compounds will be well known to anyone of ordinary skill in the art. However, with conventional nitriles production processes, it is often important that the organic feed compound is of high purity, for example at least chemical grade propylene (92-95 wt % pure) as compared with refinery grade propylene (65-75 wt % pure) in the production of acrylonitrile. This is necessary because high levels of impurities in the organic feed increase the generation of undesired organonitrile byproducts that disrupts the acrylonitrile product separation operations, degrades product quality, and generates elevated NOₓ levels. In contrast, by recycling the organonitrile byproducts to the ammoxidation reactor, the disclosed process can accept organic feeds of lower purities or blends of different grades [as available], while achieving the environmental benefits obtained from NOₓ reduction. As a result, it may not be necessary to use costly and cumbersome feed purification treatment methods to reduce the levels of impurities in the organic feeds. Similarly, oxygen and ammonia feeds of lower purity may also be acceptable.

Non-limiting examples of suitable diluents for use in the present process include C₁-C₄ alcohols (methanol, ethanol, propanol, butanol); C₁-C₄ carboxylic acids (formic, acetic, propionic) and salts thereof (ammonium, amine); C₂-C₄ ketones (acetone, methyl isobutyl ketone); low-molecular weight polyols (glycerol); certain cyclic ethers (tetrahydrofuran) and mixtures thereof. It is desirable to use a diluent that is chemically compatible with the nitriles purge stream components, have good solubility characteristics for these purge stream components, and acceptable in the ammoxidation reaction chemistry.

It is possible to use either pure, crude or mixtures of alcohols for the dilution purpose. Bio-based alcohols, such as bio-methanol, bio-ethanol, bio-propanol and bio-butanol, alcohols and glycerol from renewable feedstocks and sustainable processes, etc., are suitable feeds for the nitriles waste stream dilution. The renewable feedstocks suitable form bio-chemicals production may include biomass, lignocellulosic materials, corn, sugarcane, organic municipal waste streams, and such. The sustainable production processes may include aerobic/anaerobic digestion, fermentation, enzymatic conversions, and such.

The ratio of diluent to waste stream is not critical but typically can be from 0.01:1.0 to ≤80:1.0, such as from 0.05:1.0 to 70:1.0, for example from 0.1:1.0 to 50:1.0, all on a mass basis. In one embodiment, the diluent is an alcohol, the alcohol stream is 1 kg/hr per unit kg/hr of waste stream, or 5 kg/hr per unit kg/hr of waste stream, or 10 kg/hr per unit kg/hr of waste stream, or 20 kg/hr per unit kg/hr of waste stream.

Typically, the amount of waste stream recycled back to the ammoxidation reactor is no more than 10% by weight, such as no more than 5% by weight, for example no more than 2% by weight of the total fresh feed to the nitriles synthesis reaction zone.

In general, some level of purging of the byproducts of the ammoxidation process may be advantageous to avoid excessive build-up of non-reactive species, potentially including nitrogen-containing species. However, by diluting and recycling part of the organonitrile byproducts, the amount of NOₓ production from the purging step can be reduced to be at least 50% less, such as at least 75% less, than that of an equivalent ammoxidation process for producing the same target compound but without dilution and recycle.

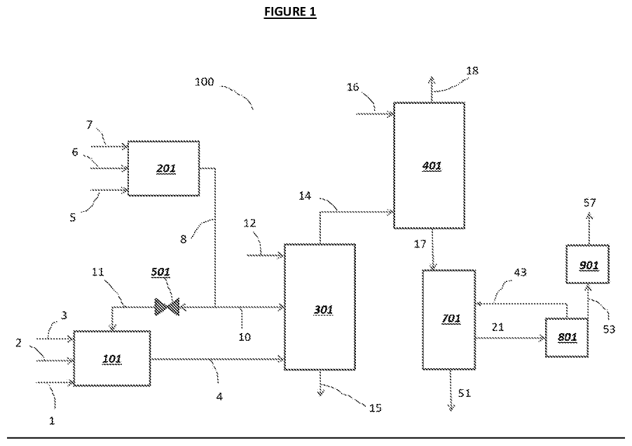

Referring to the drawings, FIG. 1 is a schematic representation of a conventional co-production facility 100 for making acrylonitrile [ACRN] and HCN. More details of such a facility can be found in U.S. Pat. No. 10,647,663, the entire contents of which are incorporated herein by reference. In the facility 100, an ammoxidation reactor system 101 catalytically converts a C₃ hydrocarbon (stream 1), such as propylene or propane, to ACRN in the presence of ammonia (stream 2) and an oxygen-containing source (stream 3). The ammoxidation reactor system 101 may include feed delivery/pre-mixing/pre-heating/distribution, a catalytic reaction zone, cyclone separators, and other auxiliary sub-systems well-described in the available ammoxidation literature.

The catalytic reaction zone in ammoxidation system 101 produces a hot, gaseous effluent (stream 4) that is rich in ACRN with byproduct acetonitrile, HCN, propionitrile, etc. Along-side the ammoxidation reactor system 101, a crude HCN product stream 8 may be produced in a stand-alone HCN synthesis reactor system 201 from a catalytic reaction between methane (stream 5), ammonia (stream 6) and oxygen-containing source (stream 7). Other examples of HCN synthesis may include methanol ammoxidation and acetonitrile conversion to HCN as well (not shown in FIG. 1). Such HCN synthesis can be run either continuously or intermittently depending on the HCN product demand.

The ammonia feed to ammoxidation reactor system 101 (stream 2) and that to HCN synthesis reactor system 201 (stream 6) may be supplied either from the same ammonia source or each may have its own dedicated source and impurity control(s). Similarly, the oxygen-containing feed to ammoxidation reactor system 101 (stream 3) and that to HCN synthesis reactor system 201 (stream 7) may be supplied either from the same oxygen source or each may have its own dedicated source. For example, the oxygen-containing source may be different in the case of air, enriched-air or 100% oxygen process utilized in HCN synthesis reactor system 201.

The crude HCN product stream 8 is routed to a quench column 301 via stream 10 along with the ammoxidation reactor gaseous stream 4. The two streams 4 and 10 may be combined or fed separately to the quench column 301. As shown in FIG. 1, the crude HCN product stream 8 may also be partially routed to ammoxidation reactor system 101 as stream 11 by regulating a flow control valve device 501. The use of routed stream 11, with components being indigenous to the ACRN production process, supplements and saves inert gases required for catalyst fluidization in 101. Depending on the extent of fluidization versus throughput load in 101, the flow control valve device 501 can be regulated to split the crude HCN product stream 8 into its routed portion stream 11 to system 101 while returning the remaining portion stream 10 to the quench system 301.

Both, the ammoxidation reactor effluent (stream 4) and the crude HCN product (stream 10) contain unconverted, excess ammonia that is removed before the products are recovered and purified. The ammoxidation reactor effluent (stream 4) along with the crude HCN product (stream 10) are processed for excess ammonia removal in a counter-current quench column system 301 using a quench liquid (stream 12). The quench liquid is acidic (low-pH) such that the excess ammonia is efficiently scrubbed out of the reactor effluent. Organic or inorganic acids (such as sulfuric acid or phosphoric acid) are used for removing ammonia at the quench column bottom (stream 15) as soluble ammonium salts. The quench liquid (stream 12) flowrate and acidity are adjusted for the combined ammonia entering in the quench column via streams 4 and 10. The excess ammonia is removed from the column 301 as ammonium salts via stream 15.

The ammonia-depleted quenched product gases (stream 14) are then passed to a counter-current absorber column system 401 where absorbent liquid (stream 16) extracts the reactor products at the bottom (stream 17) while any non-condensable and non-absorbable components are vented as off-gas (stream 18). There is no observed ammonium salt formation thereby no solid plugging at the base of absorber column system 401.

The crude product stream (17) is further processed in a downstream nitriles recovery and purification section 701 to produce high-purity ACRN along with byproduct acetonitrile and HCN. The nitriles recovery and purification section 701 comprises sequential distillative and phase separation unit operations equipped with all necessary auxiliary condensers, reboilers, recycles, pump-arounds, flowlines, etc. The nitriles recovery and purification section 701 is well-described in the available literature for acrylonitrile, acetonitrile and HCN, and therefore, not detailed herein. The output from section 701, stream 51, is collectively shown to represent individual product streams for purified HCN, acetonitrile and acrylonitrile. It is to be understood that the products, HCN, acetonitrile and acrylonitrile, are separated and purified to their desired purities in different sub-sections (not shown) of the nitriles recovery and purification system 701. For example, dry pure HCN (inhibited) is recovered from HCN Heads/Drying sub-section, while acetonitrile product is recovered from acetonitrile refining sub-section, and so on.

During the nitriles recovery and purification section 701 operation of this example, a substantial undesired organonitrile [ON] impurity stream is generated. These mid- and intermediate-boiling organonitrile impurities must be purged out of the process, which could otherwise accumulate in the final products. An impurity purge stream or a collection of such streams (represented by stream 21) is withdrawn, either continuously or intermittently, from several locations in system 701 where they are most concentrated. This purge stream 21 removal is required to maintain the steady ONs impurities balance and prevents such undesirable impurities from accumulating in the process, and ultimately in the final products.

The collected undesired organonitrile impurities purge stream 21 in the above-described process is either removed via off-gas and/or liquid concentrated stream. The nitriles purge stream 21 is fed to a recovery/disposal section 801, wherein any recoverable products of interest are stripped and returned to the process as stream 43. The residual stream 53 from section 801 then undergoes thermal destruction in thermal oxidizers [TOs] in section 901. The thermal destruction of these highly concentrated nitrogen-containing impurity purge streams lead to increased nitrogen oxides [NOₓ] emissions to the environment, and represented by off-gas stream 57 in FIG. 1.

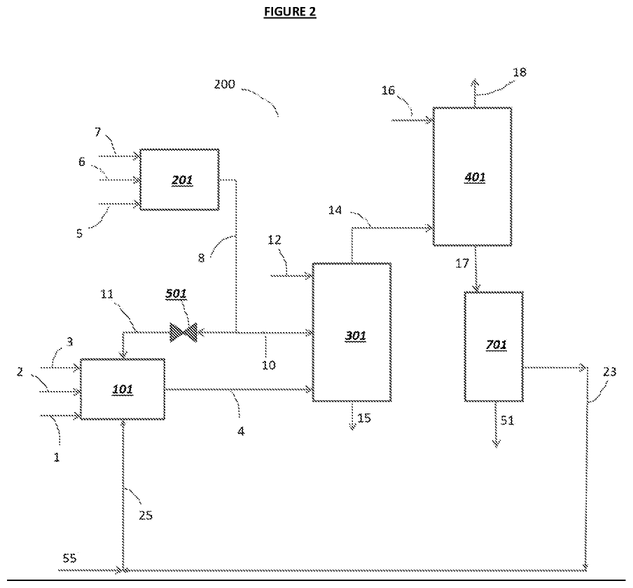

FIG. 2 is a schematic representation of a co-production facility 200 for making acrylonitrile [ACRN] and HCN according to a first embodiment of the present disclosure. The HCN-ACRN co-production facility 200 is constructed and operated like the one described in FIG. 1, except the disposal sections 801 and 901 are omitted and a waste stream 23 containing undesired organonitrile impurities is removed from the nitriles recovery and purification section 701 and recycled to the ammoxidation reactor system 101 as recycle stream 25. A separate stream 55 containing undesired organonitrile species [ONs] from another production facility may be brought in and combined in this recycle. For example, undesired ON purge streams, from an olefin hydrocyanation process for making adiponitrile, and containing butenenitriles, linear or branched pentenenitriles, glutaronitriles, valeronitrile, succinonitriles, or other undesired ON purge streams may qualify to be combined in the recycle back to the reactor 101.

The organonitrile components present in stream 23, mainly, trace HCN, acetonitrile, acrylonitrile, propionitrile and such, are inherent to the ammoxidation process. These components are unexpectedly observed to undergo further chemical conversions under the ammoxidation process condition in 101, thereby, re-utilizing the nitrogen contained in stream 25. Thus useful product yields improve from this re-routing of the nitrogen-containing stream 25.

The organonitrile waste stream 23 can either be fed continuously or can be stored in an intermediate hold vessel (not shown in FIG. 2). The feed stream 25 can be mixed with an oxygenate diluent and campaigned to the ammoxidation reactor system 101. A small portion of the stream 23, for example, less than 5% or less than 10% or less than 15%, can be diverted to the thermal oxidation system if needed or desired, say during throughput transitioning, load variations, maintenance, etc. However, for the most part, no undesired ON species are fed to the thermal oxidation system [versus as shown in FIG. 1].

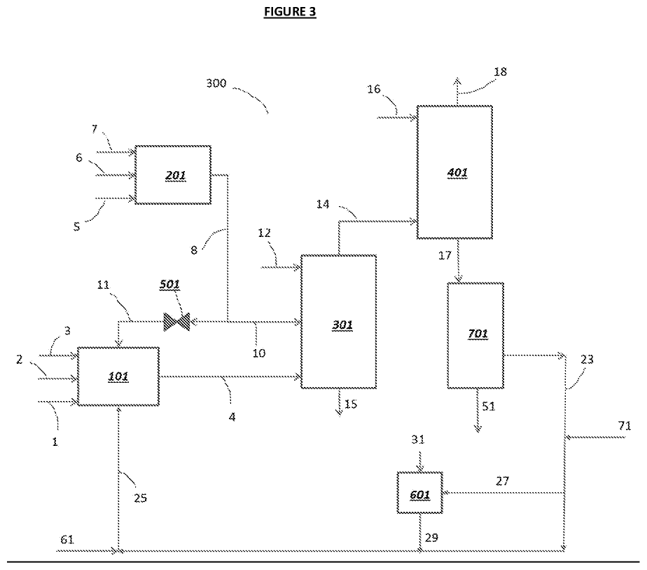

FIG. 3 is a schematic representation of a co-production facility 300 for making acrylonitrile [ACRN] and HCN according to a second embodiment of the present disclosure. The HCN-ACRN co-production facility 300 is constructed and operated like the one described in FIG. 2, except the organonitriles waste stream 23 from system 701 is diverted either partially or in totality as stream 27 to a hold/blend system 601. An oxygenate diluent stream 31 comprising an alcohol, such as methanol, ethanol, propanol, butanol or mixture thereof, is introduced to the hold/blend system 601 to blend with and dilute the undesired organonitrile purge stream 27. The resulting diluted alcohol-organonitrile stream 29 is fed to the ammoxidation reactor system 101 as stream 25.

A separate stream 61 containing undesired organonitrile species [ONs] from another production facility may be brought in and combined in this recycle. For example, undesired ON purge streams, from an olefin hydrocyanation process for making adiponitrile, and containing butenenitriles, linear or branched pentenenitriles, valeronitrile, glutaronitriles, succinonitriles, or other undesired ON purge streams may qualify to be combined in the recycle back to the reactor. Similarly, a separate stream 71 containing undesired organonitrile species [ONs] from another production facility may be brought in before dilution, if it becomes necessary to dilute such waste streams for flowability and processing ease.

The alcohol addition homogenizes the undesired organonitrile purge stream and is beneficial to free-flow of the stream between equipment without line plugging or clogging. The alcohol will also generally participate in the ammoxidation chemistry, for example, methanol contributing to HCN and ethanol contributing to acetonitrile. While there is no limit as to how much alcohol addition/dilution to use, the ammoxidation reactor system 101 operating conditions, throughput rate and cost considerations determine the ratio of dilution alcohol-to-waste stream.

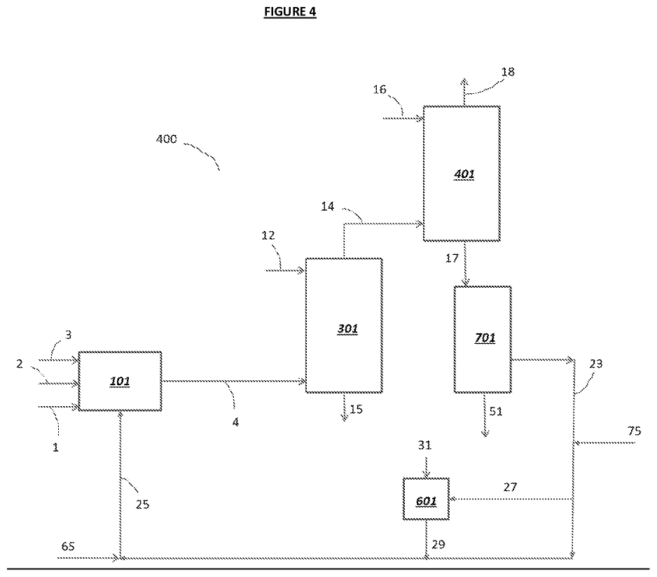

FIG. 4 is a schematic representation of a stand-alone production facility 400 for making acrylonitrile [ACRN] according to a third embodiment of the present disclosure. The facility 400 is constructed and operated like the one described in FIG. 3, except the HCN synthesis reactor system 201 and its ancillary equipment and process reams are omitted. Again, the nitriles waste stream 23 is routed back to the ammoxidation process unit 101, either undiluted or diluted via the hold/blend system 601 using an appropriate diluent. The NOₓ emissions are significantly reduced and even eliminated due to the fact that the nitriles purge stream is not thermally destructed. Instead, it is fed back to the ammoxidation reactor 101 for increased useful product yields.

A separate stream 65 containing undesired organonitrile species [ONs] from another production facility may be brought in and combined in this recycle. For example, undesired ON purge streams, from an olefin hydrocyanation process for making adiponitrile, and containing butenenitriles, linear or branched pentenenitriles, valeronitrile, glutaronitriles, succinonitriles, or other undesired ON purge streams may qualify to be combined in the recycle back to the reactor. Similarly, a separate stream 75 containing undesired organonitrile species [ONs] from another production facility may be brought in before dilution, if it becomes necessary to dilute such purge streams for flowability and processing ease.

The present process will; now be more particularly described with reference to the following non-limiting Examples.

### Comparative Example 1

An organonitrile impurity purge sub-system is represented in FIG. 1 of U.S. Pat. No. 10,562,782 [hereafter referred to as '782]. In the '782 examples, the Nitriles Purge stream labeled (8) is removed from the side-stream stripper labeled (5). From the '782 compositional data of the Nitriles Purge stream labeled (8), it is apparent that this stream is a concentrated organonitrile impurities purge stream to be disposed of after the useful contents have been recovered as stream labeled (6) that is recycled back to Separation Vessel labeled (1) in '782.

Thermal destruction of each 1.0 kg/hr of '782 stream (8) using a 99.999% destruction efficiency thermal oxidizer results in, approximately, 0.0024 g/hr of NOₓ in Example 1; 0.001 g/hr of NOₓ in Example 2; and 0.0007 g/hr of NOₓ in Example 3.

About 100 kg/hr Nitriles Purge stream labeled (8) therefore emits approximately between 0.07 to 0.24 g/hr of NOₓ in the '782 examples.

### Example 1

The process of Comparative Example 1 is repeated but with part or all the purge stream being diluted with an organic oxygenate diluent and being recycled to the ammoxidation reactor. As a result, up to 0.0024 g/hr of NOₓ emissions to the environment for each kg/hr of the Nitriles purge stream are eliminated.

1. A method of producing cyano-containing compounds, the method comprising the steps of:
(a) reacting ammonia, a source of oxygen and an organic compound in an ammoxidation reactor to produce a reaction product comprising a target compound that is a cyano-containing compound selected from hydrogen cyanide and an organonitrile compound;
(b) supplying the reaction product to a separation system to recover at least part of the target compound;
(c) providing a waste stream comprising at least one further organonitrile compound different from the target compound;
(d) combining at least part of the waste stream with at least one organic oxygenate diluent compatible with an ammoxidation reaction to produce a diluted waste stream; and
(e) supplying the diluted waste stream to the ammoxidation reactor.

2. The method of claim 1, wherein at least part of the waste stream is removed from the separation system used to recover the target cyano-containing compound.

3. The method of claim 2, further comprising purging part of the product stream through a thermal oxidation device, wherein NOₓ production from the purging step is at least 50% less than that of an equivalent ammoxidation method of producing the target compound but omitting steps (c), (d) and (e).

4. The method of claim 1, wherein the separation system is supplied with reaction products from two or more ammoxidation reactors producing cyano-containing compounds each selected from the hydrogen cyanide and the organo nitrile compound.

5. The method of claim 1, wherein the target cyano-containing compound is selected from hydrogen cyanide, acetonitrile, acrylonitrile, succinonitrile, linear pentenenitrile, branched pentenenitrile, and adiponitrile.

6. The method of claim 1, wherein at least part of the waste stream is supplied from a source different from the separation system used to recover the target cyano-containing compound.

7. The method of claim 1, wherein the diluent is selected from the group consisting of C₁-C₄ alcohols and polyols, C₁-C₄ carboxylic acids, salts of C₁-C₄ carboxylic acids, C₂-C₄ ketones, C₂-C₆ ethers, and mixtures thereof.

8. The method of claim 1, wherein the diluent comprises a C₁-C₄ alcohol selected from the group consisting of methanol, ethanol, propanol, butanol, and mixtures thereof.

9. The method of claim 1, wherein the diluent comprises a C₁-C₄ carboxylic acid or salt selected from the group consisting of formic acid, acetic acid, propionic acid, ammonium salts, amine salts, and mixtures thereof.

10. The method of claim 1, wherein the diluent comprises a C₂-C₄ ketone selected from the group consisting of acetone, methyl isobutyl ketone, and mixtures thereof.

11. The method of claim 1, wherein the diluent comprises tetrahydrofuran.

12. The method of claim 1, wherein the diluent comprises glycerol.

13. The method of claim 1, wherein the diluent comprises a bio-based organic oxygenate obtained from renewable feedstocks.

14. The method of claim 13, wherein the diluent comprises bio-methanol, bio-ethanol, bio-propanol, bio-butanol, bio-glycerol, or a mixture thereof.

15. The method of claim 1, wherein a weight ratio of diluent to waste stream is from 0.01:1.0 to 80:1.0.

16. The method of claim 1, wherein a weight ratio of diluent to waste stream is from 0.05:1.0 to 70:1.0.

17. The method of claim 1, wherein a weight ratio of diluent to waste stream is from 0.1:1.0 to 50:1.0.

18. The method of claim 1, wherein the method is operated without a feed pretreatment to reduce the levels of impurities.

19. The method of claim 1, wherein the ammoxidation reactor comprises a feed is-selected from oxygen and ammonia.
